(19) United States
(12) Patent Application Publication
(10) Pub. No.: US 2023/0210972 A1
(43) Pub. Date: Jul. 6, 2023

(54) HAKIM POURCINA'S GAND ZODA-E

(71) Applicant: Cyrus 21 Century Entrepreneurship LLC, Studio city, CA (US)

(72) Inventor: Kourosh Naziri, studio city, CA (US)

(21) Appl. No.: 18/000,100

(22) PCT Filed: Jun. 5, 2020
(86) PCT No.: PCT/US2020/036478

§ 371 Date: Nov. 28, 2022

Related U.S. Application Data
(60) Provisional application No. 63/029,872, filed on May 26, 2020.

(51) Int. Cl. A61K 39/02; A61N 7/00; A61K 35/16; A61K 39/12; A61K 41/10

(52) U.S. Cl. CPC A61K 39/02; A61K 35/16; A61K 39/12; A61K 41/10; A61N 7/00; A61K 2039/521; A61N 2007/0004

(57) ABSTRACT

A general serum procedure for all of pathogens with protein coding on an outer shell of the pathogens, comprises entering dead pathogens and their dead genomes to eliminate pathogens and cancer genes in humans and animals. A device for detecting pathogens in human body, animals, plants, water bodies, and air without blood test observes the spectral signature emitting from the human body and the water to match for any pathogens within the device’s database and uses electrostatic properties of the pathogens and their genomes to catch them.

### BACKGROUND

Traditional treatment of diseases, caused by pathogens, have searched remedies by a variety of ways, such as creating serum from animals such as horses, or working at genome level. My inventions comprising detection and scanning for pathogens by their spectral signature which requires no blood test. Each molecule or pathogen has a unique spectral signature. I have approached the treatment problem by the electrostatic properties of the pathogens. All known pathogens are negatively charged. The genes causing cancer either have positive or negative charges. By applying DC voltage at proper places by my invented devices, we can catch and take these pathogens and Genes out of body of a human, an animal, or take pathogens out of water bodies, and air in the living environment.

I have also invented an innovating procedure, which I call Local Loop Back. A small sample of 1 mL of patient’s blood is taken and the pathogens within that sample are killed. Then, the dead pathogens are injected or looped back along with a donor’s healthy blood of the same type of patient’s blood to the same patient. The advantage of this procedure is that the leukocytes encounter the dead pathogens and eliminate it, and in this way the leukocytes will learn the protein structure of the pathogen, inside the body of the patient. Now that leukocytes have learned this matter, they are ready to combat the live pathogens. This could be vaccine for pathogens. Making serum is also similar but a little more complicated. Furthermore, my invention includes hyperactivating the immune system of a patient. So far there has not been any artificial way to hyperactivate immune system. I call this procedure remote loop back. A very small sample of a donor’s healthy skin tissue, as much as tip of biopsy, entered in patient’s blood stream, along with some healthy blood of a donor that is the same type of patient’s blood. Skin tissues are really harmless to the body of a patient. But since leukocytes detect a foreign cell, they attack the donor’s skin cell. This matter is communicated to immune system. The immune system then has to create more leukocytes to combat the foreign cells. For example, covid-19 attacks and kills certain type of leukocytes, but by doing this, this pathogen dies too. New and fresh supply of leukocytes is apparent here. Note: A general vaccine and serum procedure, for all of pathogens with protein coding on outer shell of pathogen, the only exceptions are very few pathogens, such as HIV virus. My deductions: the protein coding on outer shell of virus comes from host white cell hence pathogen is hard to detect, other types of pathogens than HIV virus interfere with immune systems at DNA level. This vaccine and serum procedure can’t prevent or cure these very few kinds of pathogens

So far, there has not been any remedy to combat against pathogens existing in body tissues and organs. I know of many patients who die because of returning pathogens in blood stream from infested organs. Best examples are C Diff colitis or covid-19. The ultrasound is virtually harmless to a patient’s body, but it is deadly to pathogens. This is a great way to combat pathogens such as covid-19 or C Diff Colitis, which are also known to exist in body tissues and organs, besides the blood. Ultrasound also eliminates the pathogens feeding off of body cells and creating genomes to replicate themselves. Therefore, ultrasound kills pathogens and their genomes. Best example of these kinds of pathogens is covid-19, infesting in pulmonary and digestive systems, etc. So, this method is included in my patent application. The only thing is, some anesthesia may be necessary, ultrasound causes severe pain.

### SUMMARY

My goal in this patent application, is to detect pathogens in human body, animals, plants, water bodies, and the air, without blood test or any sampling. I have also aimed to eliminate pathogens, and cancer genes in humans and animals, by creating innovating procedures. The pathogens in air, in open spaces, plants and, water bodies could also be eliminated. I have also aimed to hyperactivate body’s immune system.

My inventions comprising detection and scanning for pathogens by their spectral signature which requires no blood test. Each molecule or pathogen has a unique spectral signature. I have approached the treatment problem by the electrostatic properties of the pathogens. All known pathogens are negatively charged. The cancer genes have either positive or negative charges. By applying DC voltage at proper places by my invented devices, we can catch and take these pathogens and cancer genes out of body of a human, an animal, or take pathogens out of water bodies, and air in the living environment.

I have also invented a procedure, which I call Local Loop Back. Local loopback procedure is applied for humans and animals. A small sample of patient’s blood is taken and the pathogens within that sample are killed. Then, the dead pathogens are injected or looped back along with a donor’s healthy blood of the same type of patient’s blood to the same patient. The advantage of this procedure is that the leukocytes encounter the dead pathogen and eliminate it, and in this way the leukocytes will learn the protein structure of the pathogen, inside the body of the patient. Now that leukocytes have learned this matter, they are ready to combat the live pathogens. This could be the vaccine for pathogens. Serum is achieved the same way, but it is a little more complicated. Furthermore, my invention includes hyperactivating the immune system of a patient. I call this procedure remote loop back, which van be used for humans and animals. A very small sample of a donor’s healthy skin tissue, as much as tip of biopsy, entered in patient’s blood along with some fresh and clean blood of a donor that is the same type of patient’s blood. Skin tissues are really harmless to the body of a patient. But since leukocytes detect a foreign cell, they attack the donor’s skin cell. This matter is communicated to immune system. The immune system then, has to create more leukocytes to combat the foreign cells. For example, covid-19 attacks and kill certain types of leukocytes, but by doing this, this pathogen dies too. New and fresh supply of leukocytes is apparent here.

The ultrasound is virtually harmless to a patient’s body, but it is deadly to pathogens. Ultrasound is used for eliminating pathogens inside organs and tissues for animals and humans. This is a great way to combat pathogens such as covid-19 or C Diff Colitis, which are also known to exist in body tissues and organs, besides the blood. Ultrasound also eliminates the pathogens feeding off of body cells and creating genomes to replicate themselves. Therefore, ultrasound kills genomes of pathogens too. Best example of these kinds of pathogens is covid-19, infesting in pulmonary and digestive systems, etc. So, this method is included in my patent application. The only thing is, some anesthesia may be necessary, since ultrasound causes severe pain. For plants applying ultrasound is enough.

### DESCRIPTION

My goal in this patent application, is to detect disease causing pathogens in human body, animals, water bodies, and the air in living spaces, without blood test or any sampling. I have also aimed to eliminate disease causing pathogens, and cancer genes in humans and animals, by creating innovating procedures. The pathogens in air in living spaces, plants and, water bodies could also be eliminated. I have also aimed to hyperactivate body’s immune system.

My inventions comprising detection and scanning for disease pathogens by their spectral signature which requires no blood test. Pathogens of any disease have a unique spectral signature. The spectral signatures of all of pathogens known will be stored in a device that I call HPC Device 1H. The processor within HPC Device 1H after scanning the patient will decide if the observed spectral signature matches to any pathogens within the database. In case of a match, device HPC 1H will alert that the patient has that specific pathogen in their body. See FIG. 1 for this device. This device has an infrared sensor for focusing on say an organ. The operator will observe the monitor to see a representation of that organ is within the screen limit. If the patient’s organ is not within limits, the operator will make adjustments to the position of scanner or position of patient, till that organ is within focus of monitor. After focusing, HPC device 1H could emit Ultraviolet waves or X-rays on that focused organ. I prefer to use Ultraviolet. A 100 watts Ultraviolet emitter or 50 watts X-ray emitter will give clear spectral images, while not harming the patient’s body. Above these 2 settings for emitter power mentioned above may be harmful to patient. Below these 2 settings for emitter power mentioned above may be yielding blurry or, low quality spectral images. I prefer ultraviolet since it is less harmful than X-rays. At a 5 second Ultraviolet emission, 1000 scans taken. The spectral signature emitting from body is observed by HPC device 1H, to match for any pathogens in HPC Device 1H’s database. The organs selected for example for covid-19 are the lungs, breathing paths, lower GI, and neural paths etc. The doctor will decide what organs need to be scanned for the pathogens, as well. A scan of an entire arm from shoulder up to the border with hand is done also, to detect the pathogens within patient’s blood stream. This same scanner-detector is used to detect pathogens inside an animal’s body.

There are 2 other variations for this scanner-detector device in [0009]. HPC Device 1W is used for water, HPC Device 1A is used for air. See FIGS. 2 and 3. For these 2 devices no infrared scanner or focusing is needed. HPC Device 1A could emit Ultraviolet waves or X-rays for spectral analysis. Emitter powers are indicated in following sentences. However other power settings are possible too, but for better quality of spectrum clarity and at the same time not damaging the environment, I recommend the following power settings for emitters. I prefer to use Ultraviolet, since it is less harmful. A 10KW Ultraviolet emitter is chosen. At a 5 second emission, 1000 scans taken. The spectral signature emitting from Air is observed by this device i.e. HPC Device 1A, to match for any pathogens in HPC Device 1A’s database. HPC device 1A scans up to 2 miles in any directions. If X-rays are chosen for HPC Device 1A, the power of emitter is 5KW and can detect up to 2 miles radius. HPC Device 1W is used for water. If X-rays are chosen for HPC Device 1W the power of emitter is 50KW and can detect up to 2 miles radius. The emitter goes into water about one meter below air surface and can scan up to 2 miles in any direction in the water, on a semi sphere area. For better quality of spectral images one meter is maximum depth that emitter submerge in water. HPC Device 1W could emit Ultraviolet waves or X-rays for spectral analysis for water. I prefer to use Ultraviolet. A 100KW Ultraviolet emitter is chosen. At a 5 second emission, 1000 scans taken. The spectral signature emitting from water is observed by HPC device 1W, to match for any pathogens in HPC device 1W’s database.

Due to negative charges within pathogens, they are captured by a pair of plates attached to DC power supply, negative pole repels and positive pole plate captures the pathogens. For cleaning the water of pathogens, after detection by HPC Device 1W of [0010], I chose a pair of square cupper plates. This plate could be 1 m by 1 m and is 1 inch thick. One plate is attached to positive side of a 100 KW power supply, while the other plate is attached to negative side of same power supply. There is a distance of 1 m between two plates. Note: a great majority of pathogens gather very near surface of water. These two plates are submerged into the body of water. These two plates could be loaded inside a small boat. After 2 hours of travel by the boat, the plates are heated in a 1000° F. oven for 20-30 minutes, to kill pathogens caught in the water body. The process continues until a great majority of pathogens are eliminated.

For cleaning the air of pathogens, after detection by HPC Device 1W of [0010], I chose a pair of square cupper plates. This plate could be 1 m by 1 m and is 1 inch thick. One plate is attached to positive side of a 10 KW power supply, while the other plate is attached to negative side of same power supply. There is a distance of 1 m between two plates. These two plates could be loaded in a small car, like a pickup truck. After 2 hours of travel by said car, the plates are heated in a 1000° F. oven for 20-30 minutes, to kill pathogens caught in the air. The process continues until a great majority of pathogens are eliminated.

I have approached the treatment of disease causing, pathogens by the electrostatic properties of the pathogens. All known pathogens are negatively charged. The genes causing cancer, have either positive or negative charges. Amoebas also have both kinds i.e. some are, positively charged and other types are negatively charged. By applying DC voltage at proper plates of device discussed in this paragraph, we can catch and take these pathogens and cancer causing, genes out of body of a human, an animal, or take pathogens out of water bodies, and air in the living environment. See FIG. 4 for HPC Device 2. drive one pint of blood from patient and place it into jar, while the jar is at lower level than bed surface. This way after the nurse initiates blood draw the gravity will get the flow into jar 10. Jar 10 has an opening on top, blood flows through a phlebotomy hose into the jar 10. This continues until the jar 10′s lid is fully contacting blood. At that time the phlebotomy valve is turned, to stop blood flow into jar 10. To compensate for water loss, white cell loss, plasma loss: administer 1000 mg of vitamin C, at the same time as treatment, patient is receiving an IV solution of water, and plasma, please also see remote loop back procedure mentioned in [0021] . Jar 10 is made of an alloy of copper-aluminum. By anodizing method, that is normally used for industrial use, this jar is processed to have pores of different sizes depending on disease. A special anodizing method is necessary that I mentioned the highlights in paragraph [0014]. The lid of this jar is not usually anodized, unless we are dealing with Amoeba ailments. A dielectric material such as flexible plastics is used to make sure that the lid and jar have no contacts. The lid is connected to the negative side of DC power supply, and the jar itself is to positive side of same power supply. The power supply has two modes 25 V or 50 V, either way the total power output is 100 W for best results. For the 25 V setting, it takes about 24 hours to completely deplete the blood from pathogen considered. For 50 V setting it takes 12 hours to do the same. However, 25 V setting is healthier for patients. We are using electrostatic properties of pathogens and their genomes to catch them. Pathogens and their genomes are negatively charged for all of known pathogens. Obviously, the pathogens and their genomes will be attracted to the walls of the jar, including the bottom wall, while the lid of jar 10 repels them. As I will discuss in paragraph [0014], by anodizing, the pores inside jar 10 are twice the size of pathogen considered. For example, if the size of a hypothetical pathogen is 150 nm then pores created inside jar 10 are selected to be 300 nm. For aids and that category of diseases, the pathogens are infesting inside of some of white cells. In that case an additional jar needed that is anodized to have a size of pores twice size of contaminated white cells. Obviously, the infested white cells will be negatively charged. These infested white cells will be attracted and caught at the pores of jar 10, for this category of pathogens. For this category of pathogens, it is strongly recommended to capture infested white cells first, the contents of jar is then, poured into another jar with specific pore size of twice the size of pathogen considered. At this point the infested white cells and the considered pathogens are captured in the pores of 2 jars by electrostatic forces. The content of final jar is free of pathogens, and infested white cells. The content of this jar is poured into a blood dispenser to be entered into patient’s artery. Now it is time to kill the pathogens. So far, jar 10 no matter what pore size it has, is still connected to DC electricity, this way the electrostatic forces will not allow the pathogens to escape. After the final jar has no blood inside, then contaminated jars can be subjected to 1000° F. for about ½ hour for elimination of pathogens. Obviously, the thin plastic insulator has to be disposed of, properly. Again, a new jar or set of 2 jars in case of aids category, is used and procedure above is repeated. When HPC Device 1H of [0002] focused to scan entire of an arm from shoulder to border with hand, and spectral signature of pathogen considered does not appear on monitor then the blood is free of pathogen considered. Taking a small blood sample from patient is good but might be misleading to customary investigation by microscope. Taking, big enough blood sample for observation by microscope is probably very inconvenient. When HPC Device 1H of [0002] shows no sign of pathogens inside jar 10 then, continue with electrostatic cleansing of blood in jar 10 for 4 jars more. This is for cases that the pathogens might me hiding in odd places. Before, sending patient home, it is better to double check by proper blood test and checking by HPC Device 1H of [0002]. Also, for diseases like corona, or C Diff colitis that pathogens hide inside tissues and organs see [0015] i.e. HPC Device 3 and, HPC Device 4 of [0023].

Anodizing is a known industrial process. To custom design for this invention I have selected some special considerations to fit the purpose of catching pathogens. This way this industrial process could have a new use and hence nobility for medical science. The highlights for anodizing for this invention is as follows: I have chosen an alloy of Copper-Aluminum/doping. Reason for alloy/doping is to make pores bigger than usual, when necessary. A jar of 1pint capacity in cubic shape to be made from this alloy. Before anodizing and then shaping a metal piece of this alloy to a cubic jar, first degreasing and then Electropolishing are done to smooth the surface of plate. Also ionize the alloy to create indenting in copper-aluminum alloy metal piece, to make bigger size of pores when necessary. Without ionizing the maximum pore size possible is 750 nm. For example, when white cells are infested with aids virus, a bigger size pores are needed to match the size of infested white cells. White cells are bigger than 750 nm. This way, white cells that are infested with aids virus that are negatively charged due to presence of aids virus, when attracted by electrostatic forces, could be captured by pores on jar and kept till incinerated. Also, the more time anodizing process takes, the bigger the size of pores will be.

See FIG. 5 for HPC Device 3. This device uses electrostatic property of pathogens and their genomes to capture them. If Dr recommends or if device 1 HPCH recognizes pathogens or their genomes inside a tissue or organ, HPC Device 3 can extract pathogens or their genomes. This device comprising two needles, like syringe needles. These needles are made of an alloy of tungsten available in some specialty vendors and are 36 gauge. Both needles are 5 inches long. There are 5 small grooves at the tip of one of the two needles. These 5 grooves have a 45 degrees angle with respect to the body of needle. These 5 grooves go to the main hollow track of needle. The grooves are created by specialty vendors by fine beam lasers. This needle is attached to positive part of a power supply. The body of this needle is rough, so are the insides of the 5 grooves and the main track of needle. The reason for this roughness is that negatively charged pathogens and genomes get attracted and attached to positive side needle, we want them to stay attached and not break free. The other needle is simple, no main track or grooves inside, and has smooth surface. This needle is connected to negative part of power supply. I have chosen voltage setting on power supply to be either set to 25 or 50 volts. However, voltages between 25 V to 100 V works. Above 100 V is unhealthy for patient. Below 25 V is not effective. The power output of power supply is 100 W for 50 volts, and power output is 50 watts for 25 volts. I prefer to use 50 V option. This option is more effective. After cleansing of blood by HPC Device 2, if HPC Device 4, mentioned in [0023] could not kill pathogens inside an organ by ultrasound, then say in a month later, HPC Device 3 can pull out the pathogen and their genomes. This procedure involves incision of 2 needles mentioned above with in 2 mm of each other. The general location of pathogens and their genomes is determined by HPC Device 1H. HPC Device 3, can catch a considered pathogen and its genomes, due to electrostatic attraction between needle attached to positive side of power supply and negatively charged pathogens and genomes. A general location within 2 mm is fine for incision. Also, there is a repelling force between needle attached to negative side of power supply and pathogens or genomes. The repelling force helps the positive needle to attract the pathogen or genome better. Best is, if possible, to make incision so that pathogen or genome are located between two needles. currently finding infinitesimal pathogens and genomes is close to impossible. However, using this device, you can even pull out genomes in one piece. This procedure is relatively nonabrasive and harmless. Usually after 10 trials even genomes come out in one piece. Note: any organ treated, might need anti swelling-anti inflammation medication. Genomes and pathogens could be cultured in a lab, then frozen at -60° F. for use at a later time.

Note that best treatment for pneumonia, in my opinion is this device 3 HPC. After applying device 4 HPC mentioned in [0023], if infection still exists, by indication by HPC Device 1H, then this incision associated with device 3 HPC seems to be best of options.

Note that based on my experience, best treatment for cancer is this HPC Device 3. Advanced cases of cancer are not curable. Best course of action is to start this treatment at early stages of cancer, when cancer is at mild stages. Even medium advanced cancer can be treated by this procedure. Malignant cancers are not treated or tested using this procedure. The power supply used has an output of 50 volts and 100 watts. Any other voltage or power either won’t work or will do more damage. Start incision at appearance location of cancer. For example, say for liver usually cancerous cells create swelling on spots on liver. The spots are starting point for incision. Trick is that cancer causing genes can be either positive or negative. So, both needles are same kind as the positive side needle of this device in [0015]. Both needles have rough surfaces inside the 5 grooves. Both needles have rough surfaces inside the main track. Both needles have rough outer surfaces. The reason for roughness of surfaces has been explained before. One can see the voltage indicator on power supply to get spikes and very infinitesimal drop in voltage. This voltage drop is in microvolts. This is when you know you are catching the cancerous genes. Incision continues for about 10 hours to free most of the organ from cancerous genes. Any time spike of voltage drop is seen you know you are at the right neighborhood. So, at this neighborhood, you continue incision until no spike on power supply is observed for 5 trials. Note to look for cancerous spots, may be X-rays are still best options to look for swellings. As mentioned before completely freeing an organ from cancerous genes is not possible, but this is a very good start. Also, incision of needles into and organ for 10 hours is still relatively close to being not badly harmful. But this procedure is extremely better than conventional surgery. Also, another significant advantage of this procedure is that the needles’ damage is so small that with Dr’s care and good nutrition and rest, the biopsy marks of needles will eventually heal. This procedure may be repeated in average every 6 months. The above example was for liver but can be used for all soft tissue organs. For brain tumors after surgery is done to take out a tumor, this procedure can be done say three months after surgery. Still we are looking for spikes and voltage drop on the power supply. The incisions are done around where the tumor was located. This procedure for cancer can’t be done for bones.

For leukemia check all white cell related organs, such as spleen, thymus etc., i.e. check by HPC Device 3 and the said spike on power supply. In case of spikes, treat that organ with same device. Use remote loop back procedure mentioned in paragraph [0022], this generates some bone morrow and leukocytes to compensate and replace damaged ones. But may not be alright for advanced cancer cases. In any case the created leukocytes and bone morrow might at least reduce the need for donor bone morrows. Remote loop back procedure, discussed in [0021], is very helpful. This could be done on the same day as any cancer treatment. After treatment is done for a patient, sterilize the needles by temperatures between, 500 and 1000, degrees Fahrenheit. I chose 550° F. Note: cancer is known to advance and migrate to different parts of body, even after surgery or any other cancer treatment. I recommend that, after cancer treatment discussed in this paragraph, random check by HPC Device 3 on different parts of body to be done. More importantly a jar similar to jar 10 of [0014] of HPC Device 2 for checking blood, where the genes might be in blood migrating to all over body. Again, we are watching for voltage drop spikes on power supply of HPC Device2 or HPC Device 3 for cancer spots/ or genes floating in blood. For any cancer, specially leukemia recommended course of action is to start with device 2 HPC. We want to catch genes in blood and infested cells with genes. The jar in this case is divided into two halves. one half will connect to positive, and the other to negative side of the 100 watts, 50 volts power supply. The halves are separated with a dielectric material such as plastics. This way infested blood cells and genes can be captured. Obviously, the pores are twice the size of biggest blood cells in question. Obviously, a blood - plasma transfusion is extremely helpful, especially for leukemia. Simultaneously HPC Device 3 is used to search inside of other organs for cancer, that might not have started inflammation or swelling inside.

As an option a fine magnetic coil is wrapped around each of two needles for HPC Device 3, while a DC voltage is applied to the coil. I do not recommend coils for this device since it is somewhat harmful to patient, and also since this method is very inefficient.

Currently to take out genomes of a pathogen inside an organ, a surgeon has to use a very abrasive procedure. This abrasive procedure involves something similar to biopsy, but the surgeon has to dig out tissues by a needle so that may be genomes are pulled out. Obviously, a big bunch of cells are pulled out along with genomes. Sometimes genomes are damaged due to this biopsy kind of procedure. This procedure is not done on live patients. Again using HPC Device 3 in paragraph [0015] is superior since whole genomes could be extracted at minimal damage to patient.

I have also invented a procedure, which I call Local Loop Back. This method of making serum and vaccine, is only applicable for pathogens having a protein coding embedded in the outer shell. Covid-19 is an example of this category. Local loop back works for both serum and vaccination for covid-19. This serum or vaccine will not work for aids. A small sample of 1 mL of sick patient’s blood is taken and the pathogens within that sample are killed. Caution must be taken that the protein coding is not damaged. Then, the dead pathogens are injected through IV or looped back along with a pint of a donor’s healthy blood, that is same type as patient’s blood to patient. This is the vaccine treatment. For serum there are 4 more stages needed. In first stage above, we used completely dead pathogens along with fully dead genomes of that pathogen, when that pathogen multiplies inside of cells of organs. In the next following 4 stages, fully dead, ¾ dead, ½ dead and ¼ dead pathogens are required. Fully dead pathogens have no activity. ¼ dead pathogens are close to fully active. ½ dead pathogens, are not dangerously active. ¾ dead pathogens have very little activity. I have used lab type microwave oven to accomplish dead or fractionally dead pathogens. The 4 additional stages for serum are as follows. Stage 1: 2 days after vaccination, use a mixture of, 90% fully dead, and 10% of ¾ dead pathogens, also completely dead pathogens along with fully dead genomes of that pathogen, in cases where, that pathogen multiplies inside of cells of organs, in one pint of a healthy donor’s blood, same type as patient’s type blood. Stage 2: 2 days after stage 1 use a mixture of, 90% fully dead, 5% of ¾ dead and 5% half dead pathogens also fully dead genomes of that pathogen, when that pathogen multiplies inside of cells of organs, in one pint of a healthy donor’s blood, same as patient’s type blood. Stage 3: 2 days after stage 2 use a mixture of, 90% fully dead, 5% of ¾ dead, 5% half dead, and 20 counts of ¼ dead pathogens, also fully dead genomes of that pathogen, when that pathogen multiplies inside of cells of organs, in one pint of a healthy donor’s blood, same type as patient’s type blood. Stage 4: 2 days after stage 3 use a mixture of, 90% fully dead, 5% of ¾ dead, 5% half dead, and 20 counts of ¼ dead pathogens, also fully dead genomes of that pathogen, when that pathogen multiplies inside of cells of organs, in one pint of a healthy donor’s blood, same type as patient’s type blood. The advantage of this procedure is that the leukocytes encounter the dead pathogen and their dead genomes and eliminate them, and in this way the leukocytes will learn the protein structure of the pathogen, inside the body of the patient. Now that leukocytes have learned this matter, they are ready to combat the live pathogens. The serum takes 7 months to make the patient’s blood free of pathogen discussed. 3 months after stage 4 serum procedure if, even slightest symptoms are present, then start the serum procedure again. If the pathogen exists in the tissues of organs, obviously the disease starts again. Experience shows that after 4 additional use of serum the disease will be cured. So, the use of this serum is as follows. After the original use of serum, wait for 3 months. If the patient still has any symptoms of that disease, then 4 additional application of said serum procedure each with one month apart should cure the patient. For vaccination the pathogen comes from a diseased donor patient. For better results please see next paragraph and using a procedure that I call remote loop back. Also, fully dead genomes could be added to serum, for better results. Also, I have introduced a procedure called remote loop back in paragraph [0022], use this procedure along with serum discussed above for extremely better results. 1000 mg of vitamin C for a few days, during and, after serum injection is recommended. Vitamin C is known to encourage generating more leukocytes.

Remote loop back: my invention includes hyperactivating the immune system of a patient. I call this procedure remote loop back. This method is useful to apply at the same time as vaccination or applying serum. Vitamin C is known to increase number of leukocytes, this may be very useful. A very small sample of a healthy donor’s skin tissue, as much as tip of biopsy, entered in 1 pint of blood from a healthy donor that has the same type of patient’s blood. This pint of blood is entered into patient through IV. Skin tissues are really harmless to the body of a patient. But since leukocytes detect a foreign cell, they attack the donor’s skin cells. This matter is communicated to immune system. The immune system then has to create more leukocytes to combat the foreign cells. For example, covid-19 attacks and kill certain types of leukocytes, but by doing this attack, this pathogen dies too. The need for new and fresh supply of leukocytes is apparent here.

HPC Device 4: The ultrasound is virtually harmless to a patient’s body, but it is deadly to pathogens. This is a great way to combat pathogens such as covid-19 or C Diff Colitis, which are also known to exist in body tissues and organs, besides the blood. Ultrasound also eliminates the pathogens feeding off of body cells and creating genomes to replicate themselves. Therefore, ultrasound kills these genomes of pathogens too. Best example of these kinds of pathogens is covid-19, infesting in pulmonary systems, breathing tracks, digestive systems, etc. So, this method is included in my patent application. The only thing is, general anesthesia is necessary, since ultrasound causes severe pain. See FIG. 6 for ultrasound device that I have invented. I call this, HPC Device 4. The power output of HPC Device 4 is 100 Watts. Say you want to kill covid-19 virus and virus’s genomes infesting in few spots on lungs. Note that genomes of say covid-19 have a different spectral signature than the spectrum of whole covid-19 virus. HPC Device 1H is used to indicate the infested spots. In this example every spot of lungs is treated, one at a time. In this example metal side of the applicator is placed on top of the locations of lungs on skin surface that HPC Device 1H has indicated. You rub the metal side of applicator of HPC Device 4 in FIG. 5, against skin area above the problem area. If neuropaths are infested by pathogens, this ultrasound device is used as well. Caution: keep away from spine. general anesthesia is required for any operation associated with HPC Device 4. For each organ 2 hours of ultrasound treatment is sufficient. I recommend that cleansing the blood is done halfway when starting ultrasound treatment. This cleansing of blood was done by HPC Device 2 of [0013]. The trick is that there might be a slight chance that the virus from organs and from patient’s blood might migrate from organ to blood or vice versa. After cleansing of say lungs for covid-19, and cleansing of blood of the virus, I recommend, to use HPC Device 1H to check for signature of covid-19 and genomes, in this example. Any sign of these signatures means repeating cleansing of blood and organ. This continues until all related covid-19 spectral signatures do not show on HPC Device 1H, and also the last 4 samples of blood shows, no sign of covid-19, that is revealed by spectral signature in HPC Device 2′s jar 10 explained in [0013]. After ultrasound treatment remote loop back of [0022] may be drastically useful. Also, anti-inflammation, and anti-swelling medications are strongly recommended, specially during use of this procedure, and a few days after, by Dr’s discretion, checking for allergies for these kinds of medications; for treatment of lungs this might be crucial.

### DESCRIPTION OF DRAWINGS

FIG. 1: HPC device 1H detects pathogens in humans or animals.

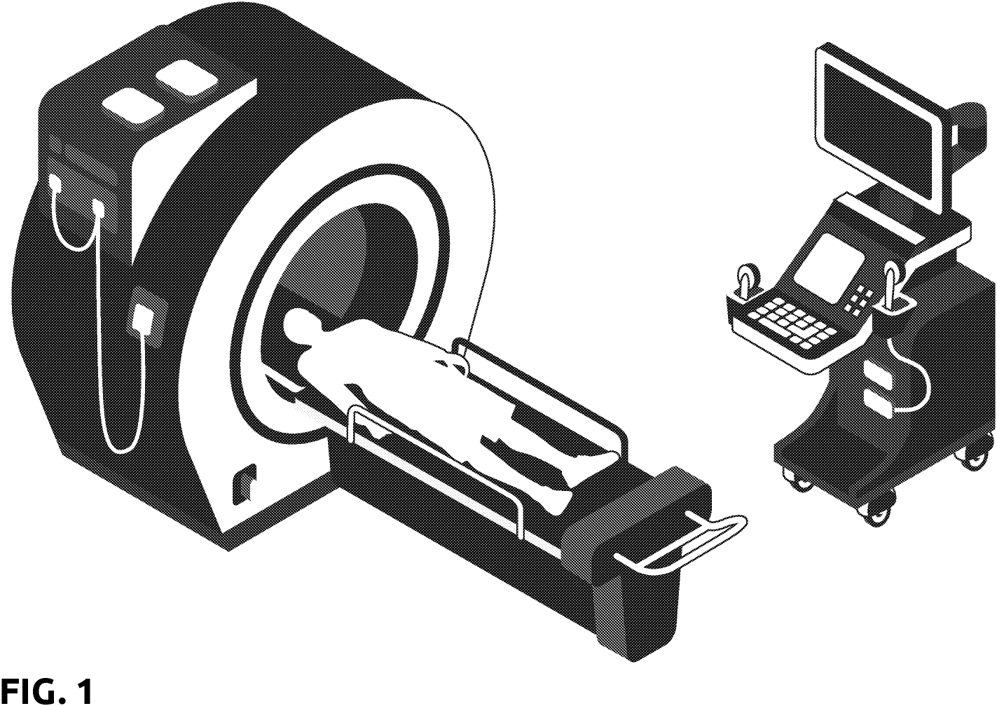

FIG. 2: HPC device 1A detects pathogens in air.

FIG. 3: HPC device 1W detects pathogens in water.

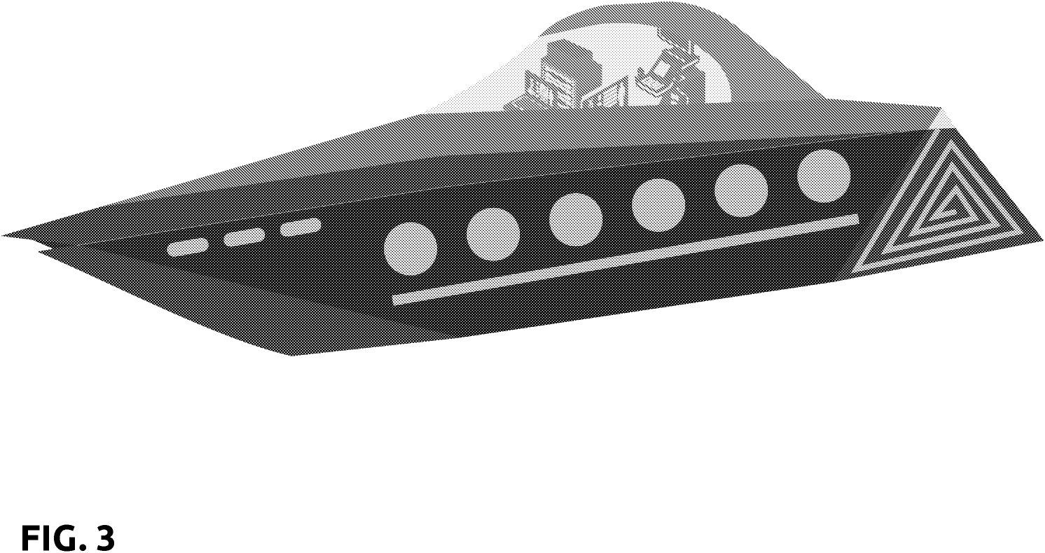

FIG. 4: HPC device 2 cleans blood from pathogens.

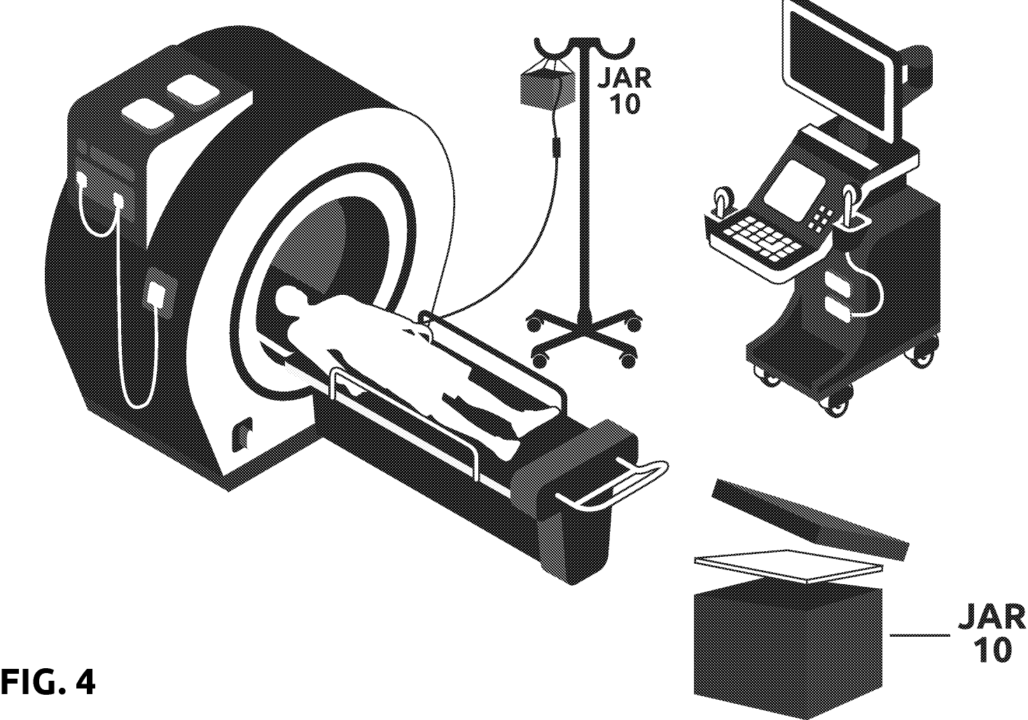

FIG. 5: HPC device 3

FIG. 6: HPC device 4

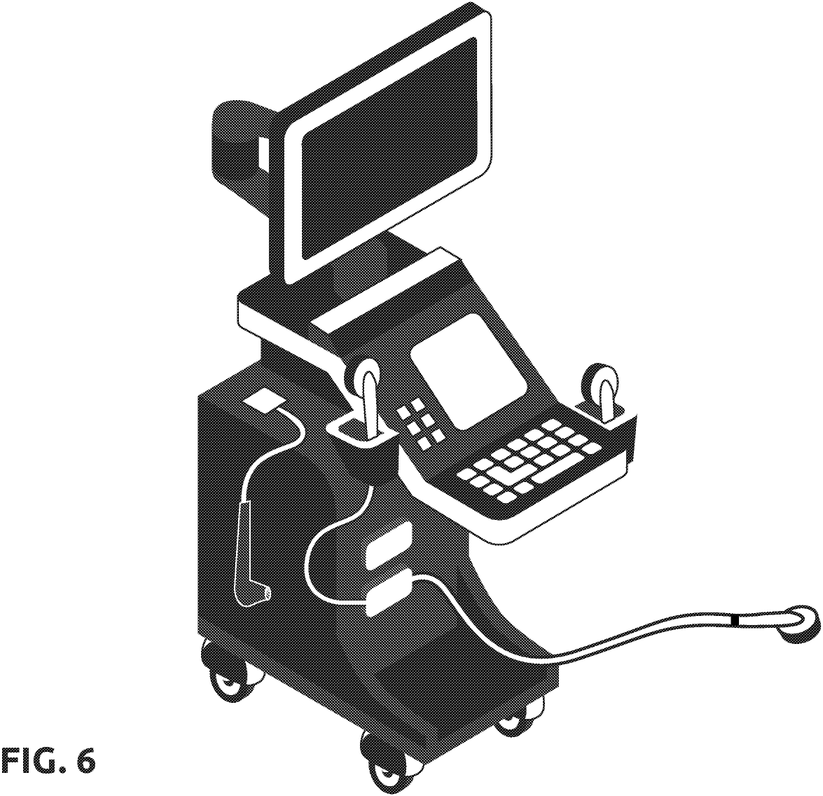

What is claimed is:

1. A vaccination that could be used for humans and animals, vaccination comprising entering dead pathogens, and their dead genomes, and a tip of biopsy amount of healthy skin tissue from a donor, through IV inside patient along with 1 pint of a donor’s healthy blood, that is same type as patient’s blood; where pathogens and their genomes, get killed by applying heat in a 100 W lab microwave oven, between 500-1000° F. for at least 20 minutes till pathogens and their genomes, would not be active anymore; this option is my favorite choice, and best of operations amongst claims mentioned in here for vaccine; protein coding of pathogens must get damaged.

2. The said killing of pathogens, and their genomes of claim 1, can be achieved wherein, an oven creating fire fueled by any hydrocarbon fuel is used.

3. The said vaccine of claim 1, can be achieved wherein, Ultraviolet is used for killing of pathogens and their genomes.

4. The said vaccine of claim 1, can be achieved wherein, X-ray is used for killing of pathogens and their genomes.

5. The said vaccine of claim 1, can be achieved wherein, Gamma rays are used for killing of pathogens and their genomes.

6. The said vaccine of claim 1, can be achieved wherein, Infrared is used for killing of pathogens and their genomes.

7. The said vaccine of claim 1, can be achieved wherein, any abrasive or harsh chemical substance is used for killing of pathogens and their genomes.

8. The said vaccine of claim 1, can be achieved wherein, any grades of bleach group currently available, is used for killing of pathogens and their genomes.

9. The said vaccine of claim 1, can be achieved wherein, any cold sterilizer, with or without vibrator machine is used for killing of pathogens and their genomes.

10. The said vaccine of claim 1, can be achieved wherein, an autoclave set between 500-1000° F. for at least 20 minutes, is used for killing of pathogens and their genomes.

11. The said vaccine of claim 1, can be achieved wherein, any concentrated antibody material is used for killing of pathogens and their genomes.

12. The said vaccine of claim 1, can be achieved wherein, any magnetic field device comprising an electronic self element with 100 watts power supply is used for killing of pathogens and their genomes.

13. The said vaccine of claim 1, can be achieved wherein, any or all combos of claims 1-12 is used, for killing of pathogens and their genomes.

14. A general serum procedure, for all of pathogens with protein coding on outer shell of pathogen, the only exceptions are very few pathogens; this serum could be used for humans and animals; serum comprising 5 stages: (a) entering dead pathogens, and their dead genomes, and a tip of biopsy amount of healthy skin tissue from a donor, through IV along with 1 pint of a donor’s healthy blood, that is same type as patient’s blood; where pathogens get processed i.e. killed by applying heat in a 100 W lab microwave oven, between 500-1000° F. till pathogens, and their genomes, would not be active anymore, (b) 2 days later, enter a mixture of assort of processed pathogens of a disease that is comprised of 90% fully dead pathogens and, a group of their dead genomes and, 10% of ¾ dead pathogens, a tip of biopsy amount of healthy skin tissue from a donor, through IV, along with 1 pint of a donor’s healthy blood, that is the same type as patient’s blood ; where fully dead pathogens and their dead genomes are processed separately, are achieved by applying heat in, a 100 W lab microwave oven, set at 500-1000° F., till pathogens and their genomes would not be active anymore, while pathogens’ protein coding is not damaged and ¾ dead pathogens are achieved by applying heat in a 100 W lab microwave oven, till pathogens would be close to inactive, while pathogens’ protein coding is not damaged (c) 2 days later, enter a mixture that is comprised of assort of processed pathogens and their genomes; mixture comprised of 90% fully dead pathogens and a group of their fully dead genomes, 5% of ¾ dead pathogens, and 5% of ½ dead pathogens, and a tip of biopsy amount of healthy skin tissue from a donor through IV, along with 1 pint of a donor’s healthy blood, that is the same type as patient’s blood; where fully dead pathogens and their dead genomes are processed separately achieved by applying 500-1000° F. heat in a 100 watts lab microwave oven, till pathogens and their genomes would not be active anymore, and ¾ dead pathogens are achieved by applying heat of 500-1000° F. in a 100 watts microwave oven in a microwave oven, till pathogens would be close to inactive, and ½ dead pathogens, are achieved by applying 500-1000° F. heat in a 100 watts microwave oven, till pathogens would not be dangerously active, (d) 2 days later enter a mixture that is comprised of assort of processed pathogens and their genomes of 90% fully dead pathogens and a group of their dead genomes, 5% of ¾ dead pathogens, 5% of ½ dead pathogens and, 20 counts of ¼ dead pathogens and, a tip of biopsy amount of healthy skin tissue from a donor through IV along with 1 pint of a donor’s healthy blood, that is same type as patient’s blood; where fully dead pathogens and their dead genomes, are achieved by processing i.e. applying heat in a lab 100 Watts microwave oven, till pathogens and their genomes would not be active anymore, ¾ dead pathogens are achieved by applying heat in a 100 Watts lab microwave oven, till pathogens, would be close to inactive, and ½ dead pathogens, are achieved by applying heat in a 100 Watts lab microwave oven, till pathogens would not be dangerously active, ¼ dead pathogens achieved by applying heat in a 100 Watts lab microwave oven, till pathogens are close to fully active, (e) 2 days later, repeat stage (d).

15. The said processing of pathogens, and their genomes of claim 14 can be achieved wherein, an oven creating fire, fueled by any hydrocarbon fuel is used.

16. The said serum of claim 14 can be achieved wherein, Ultraviolet is used for processing of pathogens and their genomes.

17. The said serum of claim 14 can be achieved wherein, X-ray is used for processing of pathogens and their genomes.

18. The said serum of claim 14 can be achieved wherein, Gamma rays are used for processing of pathogens and their genomes.

19. The said serum of claim 14 can be achieved wherein, Infrared is used for processing of pathogens and their genomes.

20. The said serum of claim 14 can be achieved wherein, any abrasive or harsh chemical substance is used for processing of pathogens and their genomes.

21. The said serum of claim 14 can be achieved wherein, any grades of bleach group currently available, is used for processing of pathogens and, their genomes.

22. The said serum of claim 14 can be achieved wherein, any cold sterilizer, with or without vibrator machine is used for killing of pathogens and their genomes.

23. The said serum of claim 14 can be achieved wherein, an autoclave set between 500-1000° F., is used for processing of pathogens and, their genomes.

24. The said serum of claim 14 can be achieved wherein, any concentrated antibody material is used for processing of pathogens and, their genomes.

25. The said serum of claim 14 can be achieved wherein, any magnetic field generator device comprising an electronic Self element with 100 watts power supply is used for processing pathogens and, their genomes.

26. The said serum of claim 14 can be achieved wherein, any or all combos of claims 14-25 is used, for processing pathogens and, their genomes.

27. HPC Device 1H is used for detection of pathogens and genomes in body; uses infrared sensor focusing on say an organ for observation ; then emits Ultraviolet on that focused organ; A 100 watts Ultraviolet emitter will give clear spectral images which is within safety region for a patient ;at a 5 second Ultraviolet emission, 1000 scans taken to observe and search for pathogens’ and their genomes’ unique spectral signature, for example for covid-19 primary organ scanned are lungs, etc.; scan of an entire arm from shoulder up to the border with hand is done also, to detect the pathogens within patient’s blood stream.

28. Said device of claim 27 is used to detect pathogens inside an animal’s body.

29. The said device of claim 27 can be achieved wherein, a 50 Watts X-ray emitter is used for detection of pathogens and their genomes.

30. Said device of claim 29 is used to detect pathogens inside an animal’s body.

31. HPC Device 2, is used for rectifying patient’s blood a pint at a time per hour; By a special anodizing method a jar is prepared; each disease pathogen requires a custom prepared jar; the DC power supply is set at 25 V with power output of 100 W for best results; in 24 hours blood is free from pathogens; this is done due to electrostatic attraction force between jar and pathogens; obviously after each use, jar has to be sterilized.

32. Said device of claim 31 could rectify blood in 12 hours, wherein DC power supply is set at 50 V and 100 W, each jar is processes in ½ of an hour.

33. For pathogens that infest within leukocytes of body the said device in claim 31 or 32 is used wherein, an additional special jar, designed for catching these leukocytes must be applied before using the said jar in claim 31, or 32.

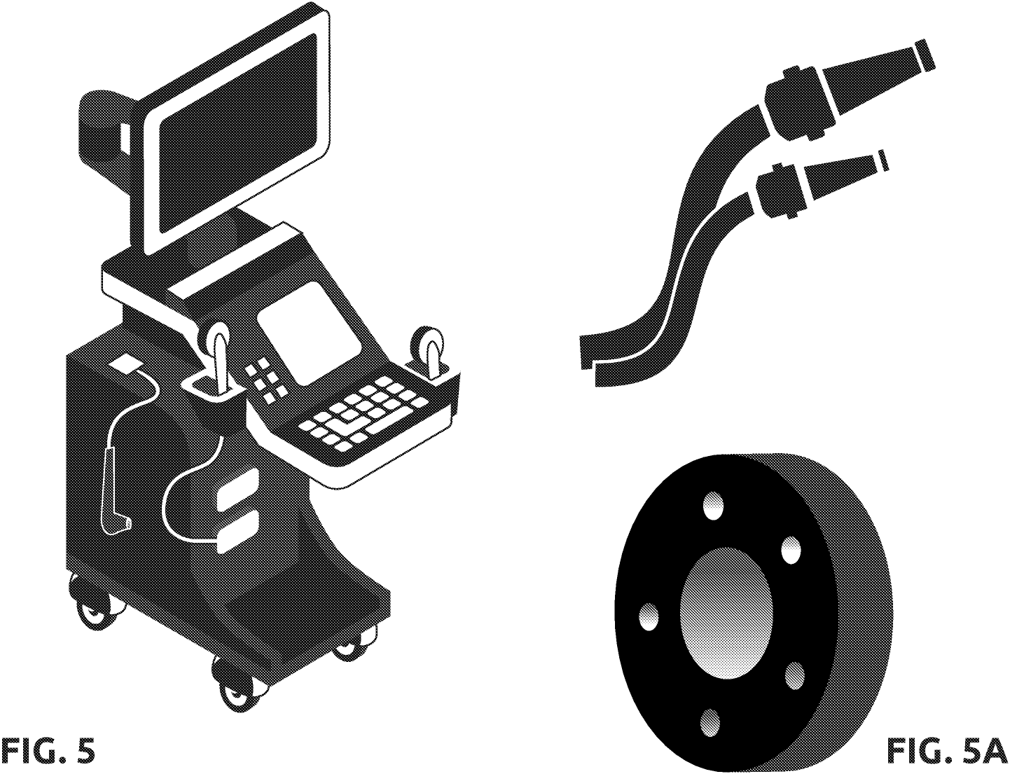

34. HPC Device 4 uses Ultrasound that is safe for humans and animals and plants, but kills pathogens and their genomes inside the body; the only thing is that, general anesthesia is necessary for animals and humans, since ultrasound causes severe pain; the power output of HPC Device 4 is 100 Watts; HPC Device 1H in claim 27 is used to indicate the infested spots; rub the metal side of applicator of HPC Device 4 in figure 5, against skin area above the problem area; If neuropaths are infested by pathogens, this ultrasound device could be used as well; warning: avoid entire spine area when using this device; for each organ 2 hours of ultrasound treatment is sufficient; anti-inflammation and anti-swelling might be crucially important, by discretion of Dr, if no allergies to drugs used, exist; Please see remote loop back of claim 37, after ultrasound treatment remote loop back may be drastically useful.

35. Pneumonia can be treated by HPC Device 4 i.e. using ultrasound treatment on lungs, using HPC device 4 in claim 34.

36. For pathogens infesting in body organs such as C Diff Colitis, or Corona virus, having genomes or not, inside an organ, first use said device in claim 31 wherein, jar used must be designed and prepared for that specific pathogen; after cleansing blood the said device in claim 34 is used to cleanse one organ at a time; when cleansing the blood is done halfway using claim 31 start ultrasound treatment in claim 34; the trick is that there might be a slight chance that the virus from organs and from patient’s blood might migrate from organ to blood or vice versa; after cleansing of say lungs for covid-19, and cleansing of blood of the virus, I recommend, to use HPC Device 1H in claim 27 to check for signature of covid-19 and genomes for example, in this example; any sign of these signatures means repeating cleansing of blood and organ, again.

37. My invention includes hyperactivating the immune system of a patient; I call this procedure remote loop back; this method is useful to apply at the same time as vaccination or applying serum in claims 1, 14; A very small sample of a healthy donor’s skin tissue, as much as tip of biopsy, entered in 1 pint of blood from a healthy donor that has the same type as patient’s blood in patient’s blood through IV; Skin tissues are really harmless to the body of a patient, but since leukocytes detect a foreign cell, they attack the donor’s skin cell, This matter is communicated to immune system; The immune system then has to create more leukocytes to combat the foreign cells; vitamin C is known to increase the number of leukocytes, this is a great help.

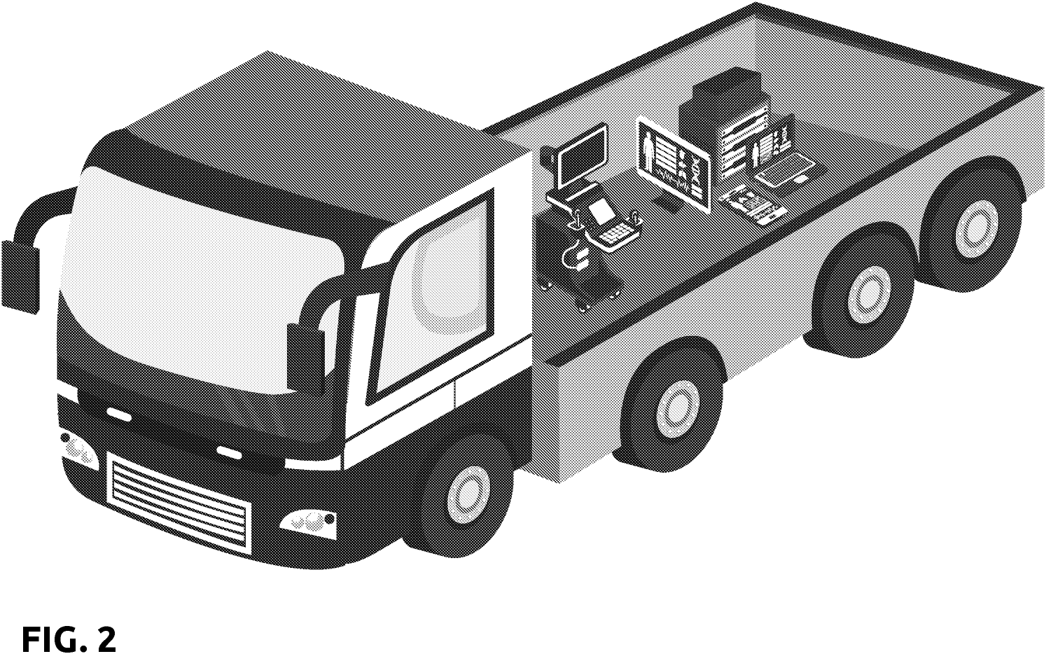

38. Device HPC 1A is used for detection of pathogens in air, see FIG. 2; said device is similar to claim 27 wherein, infrared scanner is omitted and a 10KW Ultraviolet emitter is only chosen; at a 5 second emission, 1000 scans taken, the spectral signature emitting from air is observed by HPC device 1A, to match for any pathogens in device HPC 1A’s database; HPC device 1A scans up to 2 miles in any directions.

39. Said device in claim 38 is used wherein, X-rays are chosen instead of ultraviolet; If X-rays are chosen for device HPC 1A the power of emitter is 5KW and can detect up to 2 miles radius.

40. For cleaning the air of pathogens, after detection by device HPC 1A in claim 38, I chose a pair of square cupper plates, this plate could be 1 m by 1 m and is 1 inch thick; One plate is attached to positive side of a 10 KW DC power supply, while the other plate is attached to negative side of same power supply, There is a distance of 1 m between two plates, these two plates could be loaded into a small pickup truck; After 2 hours of travel by said car, the plates are heated in a 1000° F. oven for 20-30 minutes, to kill pathogens caught from air.

41. For said device in claim 40 wherein, a magnetic field generator with strength of 100 mega Henries, with 100 windings and 10 K Amperes DC voltage could be used as an alternative; instead of DC power supply to plates, DC power is attached to windings; obviously the polarity of DC supply at windings is such that to attract the negatively charged pathogens.

42. Device HPC 1W is used for detecting pathogens in water; The emitter goes into water about one meter below air surface and can scan up to 2 miles in any direction in the water, on a semi sphere area; For better quality of spectral images one meter is maximum depth that emitter can go into water; A 100KW Ultraviolet emitter is chosen; At a 5 second emission, 1000 scans taken; The spectral signature emitting from water is observed by HPC device 1W, to match for any pathogens in HPC device 1W’s database.

43. Said device in claim 42 wherein, X-rays instead of ultraviolet is used; power of emitter is 50KW and can detect up to 2 miles radius.

44. For cleaning a water body of pathogens, after detection by HPC Device 1W in claims, 41 or 42 I chose a pair of square cupper plates, this plate could be 1 m by 1 m and is 1 inch thick; One plate is attached to positive side of a 100 KW DC power supply, while the other plate is attached to negative side of same power supply, there is a distance of 1 m between two plates; Note: a great majority of pathogens gather very near surface of water, these two plates are submerged into the body of water, these two plates could be loaded into a small boat; After 2 hours of travel by the boat, the plates are heated in a 1000° F. oven for 20-30 minutes, to kill pathogen caught in the water body; The process continues until a great majority of pathogens are eliminated.

45. For said device in claim 44 wherein, a magnetic field generator with strength of 10 mega Henries, with 100 windings and 1 K Amperes DC voltage could be used as an alternative; instead of DC power supply to plates, DC power is attached to windings; obviously the polarity of DC supply at windings is such that to attract the negatively charged pathogens.

46. See FIG. 5 for HPC device 3, this device uses electrostatic property of pathogens and their genomes to capture them, If Dr recommends or if HPC device 1H of claim 27 recognizes pathogens or their genomes inside a tissue or organ, device 3 HPC can extract pathogens or their genomes, this device comprising two needles, like syringe needles, these needles are made of an alloy of tungsten, are 36 gauge and are 5 inches long; There are 5 small grooves at tip of one of the needles, These 5 grooves have a 45 degrees angle with respect to the body of needle going in, and leading to main hollow track of syringe like needle; this needle is attached to positive part of a power supply that can have DC voltages between 25-100 volts; The body of this needle is rough, so are the insides of the 5 grooves and the main track of needle; The reason for this roughness is that negatively charged pathogens and genomes get attracted and attached to positive side needle, we want them to stay attached and not break free; the other needle is simple, no main track or grooves inside, and has smooth surface and is connected to negative part of power supply; best mode of operation is 50 volts, power output is 100 watts for 50 volts, this option is more effective; After cleansing of blood by device 2 HPC in claim 31, if HPC device 4 of claim 34 could not kill pathogens inside an organ by ultrasound, then device 3 HPC can pull out the pathogen and their genomes; this procedure involves incision of 2 needles mentioned above with in 2 mm of each other, the general location of pathogens and their genomes is determined by HPC device 1H of claim 27; also, positive side needle attracts while negative side repels mostly to positive side needle due to electrostatic forces ; usually after 10 trials even genomes come out in one piece. Genomes and pathogens could be cultured in a lab, then frozen at -60° F. for use at a later time.

47. Use said device of claim 46 for pneumonia, if said device in claim 34 does not effectively eliminate the infection; please use all cautions indicated in claim 34.

48. Use said device of claim 46 for diseases caused by amoeba wherein, both needles are of the kind as used for positive side of claim 46, since amoebas could be either positive or negatively charged; In case of blood contamination device in claim 31 could be used; trick is that negatively charged amoeba gets attracted to positive side i.e. body of jar in claim 31, and positively charged amoeba get caught by lid of said jar; obviously the negative and positive poles of power supply can be interchanged by Dr’s decision, depending on situation.

49. Best treatment for cancer genes is this HPC device 3 in claim 46, and HPC device 2 in claim 31. usually advanced cases of cancer are not curable, but it might be useful to try. Even medium advanced cancer can be treated by this procedure; the power supply used has an output of 50 volts and 100 watts, any other voltage or power either won’t work or will do more damage; start incision at appearance location of cancer, for example, say for liver usually cancerous cells create swelling on spots on liver, spots are starting point for incision; trick is that cancer causing genes can be either positive or negative so, both needles are same kind as the positive side needle of this device; One can see the voltage indicator on power supply to get spikes and very infinitesimal drop in voltage in micro volts, but it is visible, this is when you know you are catching the cancerous genes; Incision continues for about 10 hours to free most of the organ from cancerous genes, any time spike of voltage drop is seen you know you are at the right neighborhood so, you continue incision until no spike on power supply is observed for 5 trials on that very spot; may be X-rays are still best options to look for swellings, completely freeing an organ from cancerous genes is not possible, but this is a very good start; incision of needles into an organ for 10 hours is still relatively close to being not badly harmful, but this procedure is extremely better than conventional surgery; this procedure may be repeated in average every 6 months, The above example was for liver but can be used for all soft tissue organs; Note: cancer is known to advance and migrate to different parts of body, even after surgery or any other cancer treatment; I recommend that, after cancer treatment, random check by HPC device 3 on different parts of body to be done; again we are watching for voltage drop spikes on power supply of HPC device 3 for cancer gene spots. More importantly HPC device 2 for checking blood, where the genes might be in blood migrating to all over body. Again, we are watching for voltage drop spikes on power supply of HPC device 3, or HPC device 2 of claims 31 and, 42 for cancer gene spots/ or genes floating in blood. For any cancer, specially leukemia recommended course of action is to start with device 2 HPC; we want to catch genes in blood and infested cells with genes. The jar in this case is divided into two halves. one half will connect to positive, and the other to negative side of the 100 watts, 50 volts power supply. The halves are separated with a dielectric material such as plastics. This way infested blood cells and genes can be captured. Obviously, the pores are twice the size of biggest blood cells in question. Simultaneously device 3 HPC is used to search inside of cancer, stricken organs, in case of detecting genes in blood, blood or plasma rather is strongly recommended; Said procedure in this paragraph is used for brain tumors as well, say three months after surgery when tumor taken out; Still we are looking for spikes and voltage drop on the power supply, the incisions are done around where the tumor was located.

50. For leukemia use the procedure in claim 49 wherein, all of soft tissue white cell related organs, such as spleen, thymus etc. should be treated; use remote loop back procedure mentioned in claim 37 after treatment, this creates some bone morrow and leukocytes to compensate and replace damaged ones; created bone morrow might at least reduce the need for donor bone morrows.

51. For said device on claims 31, wherein, a magnetic field generator with strength of 2 kilo Henries could be used as an alternative; obviously the polarity at windings is such that to attract the negatively charged pathogens.

52. For said device on claims 32, wherein, a magnetic field generator with strength of 2 kilo Henries could be used as an alternative; obviously the polarity at windings is such that to attract the negatively charged pathogens.

53. For said device on claims 46 wherein, a magnetic field generator with strength of 2 kilo Henries could be used as an alternative; windings to be wrapped around said needles; instead of DC power supply attached to needles, DC power is attached to windings; obviously longer needles are required; obviously the polarity of DC power supply at windings is such that to attract the negatively charged pathogens.

54. For said device on claim 49 a magnetic field generator with strength of 2 kilo Henries could be used as an alternative; windings to be wrapped around said needles; instead of DC power supply attached to needles, DC power is attached to windings; obviously longer needles are required; obviously the polarity of DC power supply at windings is such that to attract the negatively charged pathogens.

55. For said procedure in claim 1 don’t introduce the said tip of biopsy amount of skin tissue in patient’s blood stream.

56. The said killing of pathogens, and their genomes of claim 55, can be achieved wherein, an oven creating fire fueled by any hydrocarbon fuel is used.

57. The said vaccine of claim 55, can be achieved wherein, Ultraviolet is used for killing of pathogens and their genomes.

58. The said vaccine of claim 55, can be achieved wherein, X-ray is used for killing of pathogens and their genomes.

59. The said vaccine of claim 55, can be achieved wherein, Gamma rays are used for killing of pathogens and their genomes.

60. The said vaccine of claim 55, can be achieved wherein, Infrared is used for killing of pathogens and their genomes.

61. The said vaccine of claim 55, can be achieved wherein, any abrasive or harsh chemical substance is used for killing of pathogens and their genomes.

62. The said vaccine of claim 55, can be achieved wherein, any grades of bleach group currently available, is used for killing of pathogens and their genomes.

63. The said vaccine of claim 55, can be achieved wherein, any cold sterilizer, with or without vibrator machine is used for killing of pathogens and their genomes.

64. The said vaccine of claim 55, can be achieved wherein, an autoclave set between 500-1000° F. for at least 20 minutes, is used for killing of pathogens and their genomes.

65. The said vaccine of claim 55, can be achieved wherein, any concentrated antibody material is used for killing of pathogens and their genomes.

66. The said vaccine of claim 55, can be achieved wherein, any magnetic field device comprising an electronic self element with 100 watts power supply is used for killing of pathogens and their genomes.

67. The said vaccine of claim 55, can be achieved wherein, any or all combos of claims 55-66 is used, for killing of pathogens and their genomes.

68. For said procedure in claim 1 don’t introduce the said dead genomes in patient’s blood stream.

69. The said killing of pathogens, and their genomes of claim 68, can be achieved wherein, an oven creating fire fueled by any hydrocarbon fuel is used.

70. The said vaccine of claim 68, can be achieved wherein, Ultraviolet is used for killing of pathogens and their genomes.

71. The said vaccine of claim 68, can be achieved wherein, X-ray is used for killing of pathogens and their genomes.

72. The said vaccine of claim 68, can be achieved wherein, Gamma rays are used for killing of pathogens and their genomes.

73. The said vaccine of claim 68, can be achieved wherein, Infrared is used for killing of pathogens and their genomes.

74. The said vaccine of claim 68, can be achieved wherein, any abrasive or harsh chemical substance is used for killing of pathogens and their genomes.

75. The said vaccine of claim 68, can be achieved wherein, any grades of bleach group currently available, is used for killing of pathogens and their genomes.

76. The said vaccine of claim 68, can be achieved wherein, any cold sterilizer, with or without vibrator machine is used for killing of pathogens and their genomes.

77. The said vaccine of claim 68, can be achieved wherein, an autoclave set between 500-1000° F. for at least 20 minutes, is used for killing of pathogens and their genomes.

78. The said vaccine of claim 68, can be achieved wherein, any concentrated antibody material is used for killing of pathogens and their genomes.

79. The said vaccine of claim 68, can be achieved wherein, any magnetic field device comprising an electronic self element with 100 watts power supply is used for killing of pathogens and their genomes.

80. The said vaccine of claim 68, can be achieved wherein, any or all combos of claims 55-66 is used, for killing of pathogens and their genomes.

81. For said procedure in claim 1 don’t introduce the said dead genomes, or the skin tissues said in claim 1, in patient’s blood stream.

82. The said killing of pathogens, and their genomes of claim 81, can be achieved wherein, an oven creating fire fueled by any hydrocarbon fuel is used.

83. The said vaccine of claim 81, can be achieved wherein, Ultraviolet is used for killing of pathogens and their genomes.

84. The said vaccine of claim 81, can be achieved wherein, X-ray is used for killing of pathogens and their genomes.

85. The said vaccine of claim 81, can be achieved wherein, Gamma rays are used for killing of pathogens and their genomes.

86. The said vaccine of claim 81, can be achieved wherein, Infrared is used for killing of pathogens and their genomes.

87. The said vaccine of claim 81, can be achieved wherein, any abrasive or harsh chemical substance is used for killing of pathogens and their genomes.

88. The said vaccine of claim 81, can be achieved wherein, any grades of bleach group currently available, is used for killing of pathogens and their genomes.

89. The said vaccine of claim 81, can be achieved wherein, any cold sterilizer, with or without vibrator machine is used for killing of pathogens and their genomes.

90. The said vaccine of claim 81, can be achieved wherein, an autoclave set between 500-1000° F. for at least 20 minutes, is used for killing of pathogens and their genomes.

91. The said vaccine of claim 81, can be achieved wherein, any concentrated antibody material is used for killing of pathogens and their genomes.

92. The said vaccine of claim 81, can be achieved wherein, any magnetic field device comprising an electronic self element with 100 watts power supply is used for killing of pathogens and their genomes.

93. The said vaccine of claim 81, can be achieved wherein, any or all combos of claims 81-92 is used, for killing of pathogens and their genomes.

94. For said procedure in claim 14 don’t introduce the said tip of biopsy amount if skin tissue in patient’s blood stream.

95. The said processing of pathogens, and their genomes of claim 94 can be achieved wherein, an oven creating fire, fueled by any hydrocarbon fuel is used.

96. The said serum of claim 94 can be achieved wherein, Ultraviolet is used for processing of pathogens and their genomes.

97. The said serum of claim 94 can be achieved wherein, X-ray is used for processing of pathogens and their genomes.

98. The said serum of claim 94 can be achieved wherein, Gamma rays are used for processing of pathogens and their genomes.

99. The said serum of claim 94 can be achieved wherein, Infrared is used for processing of pathogens and their genomes.

100. The said serum of claim 94 can be achieved wherein, any abrasive or harsh chemical substance is used for processing of pathogens and their genomes.

101. The said serum of claim 94 can be achieved wherein, any grades of bleach group currently available, is used for processing of pathogens and, their genomes.

102. The said serum of claim 94 can be achieved wherein, any cold sterilizer, with or without vibrator machine is used for killing of pathogens and their genomes.

103. The said serum of claim 94 can be achieved wherein, an autoclave set between 500-1000° F., is used for processing of pathogens and, their genomes.

104. The said serum of claim 94 can be achieved wherein, any concentrated antibody material is used for processing of pathogens and, their genomes.

105. The said serum of claim 94 can be achieved wherein, any magnetic field generator device comprising an electronic Self element with 100 watts power supply is used for processing pathogens and, their genomes.

106. The said serum of claim 94 can be achieved wherein, any or all combos of claims 94-105 is used, for processing pathogens and, their genomes.

107. For said procedure in claim 14 don’t introduce the said dead genomes in patient’s blood stream.

108. The said processing of pathogens, and their genomes of claim 107 can be achieved wherein, an oven creating fire, fueled by any hydrocarbon fuel is used.

109. The said serum of claim 107 can be achieved wherein, Ultraviolet is used for processing of pathogens and their genomes.

110. The said serum of claim 107 can be achieved wherein, X-ray is used for processing of pathogens and their genomes.

111. The said serum of claim 107 can be achieved wherein, Gamma rays are used for processing of pathogens and their genomes.

112. The said serum of claim 107 can be achieved wherein, Infrared is used for processing of pathogens and their genomes.

113. The said serum of claim 107 can be achieved wherein, any abrasive or harsh chemical substance is used for processing of pathogens and their genomes.

114. The said serum of claim 107 can be achieved wherein, any grades of bleach group currently available, is used for processing of pathogens and, their genomes.

115. The said serum of claim 107 can be achieved wherein, any cold sterilizer, with or without vibrator machine is used for killing of pathogens and their genomes.

116. The said serum of claim 107 can be achieved wherein, an autoclave set between 500-1000° F., is used for processing of pathogens and, their genomes.

117. The said serum of claim 107 can be achieved wherein, any concentrated antibody material is used for processing of pathogens and, their genomes.

118. The said serum of claim 107 can be achieved wherein, any magnetic field generator device comprising an electronic Self element with 100 watts power supply is used for processing pathogens and, their genomes.

119. The said serum of claim 107 can be achieved wherein, any or all combos of claims 107-118 is used, for processing pathogens and, their genomes.

120. For said procedure in claim 14 don’t introduce the said tip of biopsy amount if skin tissue, or dead genomes in patient’s blood stream.

121. The said processing of pathogens, and their genomes of claim 120 can be achieved wherein, an oven creating fire, fueled by any hydrocarbon fuel is used.

122. The said serum of claim 120 can be achieved wherein, Ultraviolet is used for processing of pathogens and their genomes.

123. The said serum of claim 120 can be achieved wherein, X-ray is used for processing of pathogens and their genomes.

124. The said serum of claim 120 can be achieved wherein, Gamma rays are used for processing of pathogens and their genomes.

125. The said serum of claim 120 can be achieved wherein, Infrared is used for processing of pathogens and their genomes.

126. The said serum of claim 120 can be achieved wherein, any abrasive or harsh chemical substance is used for processing of pathogens and their genomes.

127. The said serum of claim 120 can be achieved wherein, any grades of bleach group currently available, is used for processing of pathogens and, their genomes.

128. The said serum of claim 120 can be achieved wherein, any cold sterilizer, with or without vibrator machine is used for killing of pathogens and their genomes.

129. The said serum of claim 120 can be achieved wherein, an autoclave set between 500-1000° F., is used for processing of pathogens and, their genomes.

130. The said serum of claim 120 can be achieved wherein, any concentrated antibody material is used for processing of pathogens and, their genomes.

131. The said serum of claim 120 can be achieved wherein, any magnetic field generator device comprising an electronic Self element with 100 watts power supply is used for processing pathogens and, their genomes.

132. The said serum of claim 120 can be achieved wherein, any or all combos of claims 120-131 is used, for processing pathogens and, their genomes.
